(19) United States
(12) Patent Application Publication
(10) Pub. No.: US 2007/0092477 A1
(43) Pub. Date: Apr. 26, 2007

(54) Cleaning compositions

(75) Inventors: Tak Wai Cheung, Montvale, NJ (US); Andrew Courtney, Montvale, NJ (US); Edward Fu, Montvale, NJ (US); Steven Wu, Montvale, NJ (US)

Correspondence Address: NORRIS, MCLAUGHLIN & MARCUS, 875 THIRD AVE, 18TH FLOOR, NEW YORK, NY, 10022, US

(73) Assignee: RECKITT BENCKISER INC., Parsippany, NJ (US)

(21) Appl. No.: 10/595,872

(22) PCT Filed: Nov. 18, 2004
(86) PCT No.: PCT/GB04/04860

§ 371 Date: Nov. 30, 2006

(30) Foreign Application Priority Data
Nov. 21, 2003 (GB) 0327091.5

(51) Int. Cl. A61K 8/46

(52) U.S. Cl. 424/70.24

(57) ABSTRACT

Improved treatment blocks useful in the treatment of lavatory appliances, particularly toilets are provided. The improved treatment blocks are solid block compositions which comprise at least one detersive surfactant, a diester constituent, and one or more further optional constituents, including bleach constituents. The improved treatment blocks provide improved processing and handling characteristics. Methods of producing the solid block composition and treatment blocks therefrom, as well as methods of use are also disclosed.

The present invention relates to improved solid treatment block compositions useful for providing an active treatment composition to a sanitary appliance, e.g., a toilet or urinal. More specifically the present invention relates to improved solid block cleaning compositions containing diester compounds used for such purposes.

Solid treatment block have found widespread use in the cleaning and/or disinfecting treatment of sanitary appliances as, once installed they require little or no user intervention during their effective service life. Such solid treatment block compositions are considered to operate in an automatic fashion and their effective functioning is dependent in great part upon their composition, their dissolution characteristics when contacted with water and their placement within the sanitary appliance which they are used to treat. Typically such solid treatment block compositions are used in either one of two modes, either as an “ITC” or “in the cisern” mode, or as an “ITB” or “in the bowl” mode. In the former the solid treatment block composition is placed in water supply tank, also known as the cistern or toilet tank wherein it is expected to dissolve over a period of time and thus deliver active cleaning and/or disinfecting constituents to the water present in the cistern which is periodically used to flush the toilet bowl or other sanitary appliance, e.g., a urinal. Such a solid treatment block composition may be supplied to the interior of the cistern as a tablet or other self supporting shape, or alternately the solid treatment block composition may be provided in a container or cage, or as part of a dispensing device, from which the active cleaning and/or disinfecting constituents are delivered to the water present in the cistern. In the latter, the solid treatment block composition is placed within the bowl, typically supported by a device, cage, or even a simple bent wire such that the active cleaning and/or disinfecting constituents are contacted with water flushed into the sanitary appliance, especially the bowl of a toilet, or the interior of a urinal. In such an installation it is expected that a part of the solid treatment block composition is dissolved with each flush of water passing though the device such that an amount of active cleaning and/or disinfecting constituents are dispensed to the toilet bowl, urinal, etc.

The art is replete with may forms of solid treatment block compositions which find use either as ITB or ITC type compositions. Examples of such solid treatment block compositions include those described in the following: U.S. Pat. No. 4,246,129; U.S. Pat. No. 4,269,723; U.S. Pat. No. 4,043,931; U.S. Pat. No. 4,460,490; U.S. Pat. No. 4,722,802; U.S. Pat. No. 4,820,449; U.S. Pat. No. 5,342,550; U.S. Pat. No. 5,562,850; U.S. Pat. No. 5,711,920; U.S. Pat. No. 5,759,974; U.S. Pat. No. 5,939,372; U.S. Pat. No. 6,001,789 as well as U.S. Pat. No. 6,294,510. Each of these patents disclosed solid treatment block compositions which provide specific technical benefits, or overcome specific technical shortcomings which were hithero known to the art until the time of the respective invention. For example, various processing shortcomings are known from the manufacture of such blocks, or from the dissolution characteristics of such blocks as are described in these patents or which are otherwise known to the relevant art.

Thus, while these solid treatment block compositions are useful and provide certain advantageous features there is nonetheless a real and continuing need in the art for further solid treatment block compositions which are effective in the treatment of sanitary appliances both in an ITB and/or in an ITC mode. There also remains a real and urgent need in the art for such improved solid treatment block compositions which provide improved manufacturing effects, improved handling effects subsequent to the manufacture of such solid treatment block compositions, as well as improved block stability effects of such solid treatment block compositions particularly when used within a device such as in an ITB or ITC device installed in a toilet or other sanitary appliance.

Accordingly it is an object of the present invention to provide an improved solid treatment block composition useful as an ITB or ITC device installed in a toilet or other sanitary appliance. Such a solid treatment block composition operates to provide a cleaning and bleaching effect (preferably both cleaning and bleaching effect) to sanitary appliances within which they are used.

It is a further object of the invention to provide improved processes for the manufacture of the aforesaid solid treatment block compositions.

It is a yet further object of the invention which exhibits improved handling characteristics subsequent to the manufacture of the aforesaid solid treatment block compositions, especially prior to their use of solid blocks formed therefrom as an ITB or ITC device installed in a toilet or other sanitary appliance.

It is a still further object of the invention to provide an improved solid treatment block composition useful as an ITB or ITC device in the form of a solid, self-supporting block installed in a toilet or other sanitary appliance which exhibits good delivery characteristics and dimensional stability during their use.

These and other objects of the invention will become apparent to those of ordinary skill in this art from the following detailed description.

According to one aspect of the invention there is provided a treatment block formed from a solid block composition which includes: a surfactant constituent, a diester constituent, and one or more further optional constituents.

According to a second aspect of the invention there is provided a treatment block formed from a solid block composition which includes: a surfactant-constituent, a diester constituent, a bleach constituent, and optionally one or more further constituents.

In a further aspect of the invention there is provide an improved treatment block according to the first or second aspects of the invention as recited above which exhibits good delivery characteristics and dimensional stability during their use in providing a cleaning and/or disinfecting treatment of a lavatory appliance within which they are used.

In a yet further aspect of the invention there is provided an improved treatment block according to the first or second aspects of the invention as recited above which provide improved manufacturing characteristics particularly improved extrusion characteristics and/or improved handling characteristics of treatment blocks formed from the solid block composition subsequent to their manufacture but prior to their use in a sanitary appliance.

The solid block composition of the invention necessarily comprises a surfactant constituent which comprises one or more detersive surfactants. Exemplary useful surfactants include anionic, nonionic, cationic, amphoteric, and zwitterionic surfactants, particularly those whose melting points are sufficiently high, above about 110° F., preferably above 125° F., to permit processing according to known art techniques. However, small amounts of low melting point surfactants and even liquid surfactants may be used in providing the surfactant constituent.

Exemplary useful anionic surfactants which may be used in the solid block composition of the invention can be broadly described as the water-soluble salts, particularly the alkali metal salts, of organic sulfuric acid reaction products having in their molecular structure an alkyl or alkaryl radical containing from about 8 to about 22 carbon atoms and a radical selected from the group consisting of sulfonic acid and sulfuric acid ester radicals. (Included in the term alkyl is the alkyl portion of higher acyl radicals.) Important examples of the anionic surfactants which can be employed in practicing the present invention are the sodium or potassium alkyl sulfates, especially those obtained by sulfating the higher alcohols (C₈-C₁₈ carbon atoms) produced by reducing the glycerides of tallow or coconut oil; sodium or potassium alkyl benzene sulfonates, in which the alkyl group contains from about 9 to about 15 carbon atoms, (the alkyl radical can be a straight or branched aliphatic chain); paraffin sulfonate surfactants having the general formula RSO₃ M, wherein R is a primary or secondary alkyl group containing from about 8 to about 22 carbon atoms (preferably 10 to 18 carbon atoms) and M is an alkali metal, e.g., sodium, lithium or potassium; sodium alkyl glyceryl ether sulfonates, especially those ethers of the higher alcohols derived from tallow and coconut oil; sodium coconut oil fatty acid monoglyceride sulfates and sulfonates; sodium or potassium salts of sulfuric acid esters of the reaction product of one mole of a higher fatty alcohol (e.g., tallow or coconut oil alcohols) and about 1 to 10 moles of ethylene oxide; sodium or potassium salts of alkyl phenol ethylene oxide ether sulfates with about 1 to about 10 units of ethylene oxide per molecule and in which the alkyl radicals contain from about 8 to about 12 carbon atoms; the reaction products of fatty acids esterified with isethionic acid and neutralized with sodium hydroxide where, for example, the fatty acids are derived from coconut oil; sodium or potassium salts of fatty acid amides of a methyl tauride in which the fatty acids, for example, are derived from coconut oil and sodium or potassium β-acetoxy- or β-acetamido-alkanesulfonates where the alkane has from 8 to 22 carbon atoms. Further useful anionic surfactants include those which comprise a succinate moiety.

A preferred class of anionic surfactants are linear alkyl benzene sulfonate surfactant wherein the alkyl portion contains 8 to 16 carbon atoms, and most preferably about 11 to 13 carbon atoms. According to certain particularly preferred embodiments of the invention, the solid block compositions necessarily include anionic linear alkyl benzene sulfonates containing 11, 12 or 13 carbon atoms, or salt forms thereof.

A further preferred class of anionic surfactants are olefin sulfonates, preferably alpha olefin sulfonates wherein the olefin portion contains 10 to 18 carbon atoms, and most preferably contains 14 to 16 carbon atoms. According to certain further particularly preferred embodiments, the invention the solid block compositions necessarily include alpha olefin sulfonates containing 14, 15 or 16 carbon atoms in the olefin portion or salt forms thereof.

A yet further preferred class of anionic surfactants are those which include a sulfosuccinate moiety.

In certain preferred embodiments of the block compositions the sole anionic surfactant present is an anionic linear alkyl benzene sulfonate or salt thereof.

In certain further preferred embodiments of the block compositions both an anionic linear alkyl benzene sulfonate or salt thereof as well as alpha olefin sulfonates containing 14, 15 or 16 carbon atoms in the olefin portion or salt forms thereof are necessarily present.

The detersive surfactant constituent of the solid block composition of the invention may include one or more nonionic surfactants. Practically any hydrophobic compound having a carboxy, hydroxy, amido, or amino group with a free hydrogen attached to the nitrogen can be condensed with an alkylene oxide, especially ethylene oxide or with the polyhydration product thereof, a polyalkylene glycol, especially polyethylene glycol, to form a water soluble or water dispersible nonionic surfactant compound. Further, the length of the polyethenoxy hydrophobic and hydrophilic elements may various. Exemplary nonionic compounds include the polyoxyethylene ethers of alkyl aromatic hydroxy compounds, e.g., alkylated polyoxyethylene phenols, polyoxyethylene ethers of long chain aliphatic alcohols, the polyoxyethylene ethers of hydrophobic propylene oxide polymers, and the higher alkyl amine oxides.

One class of useful nonionic surfactants include polyalkylene oxide condensates of alkyl phenols. These compounds include the condensation products of alkyl phenols having an alkyl group containing from about 6 to 12 carbon atoms in either a straight chain or branched chain configuration with an alkylene oxide, especially an ethylene oxide, the ethylene oxide being present in an amount equal to 5 to 25 moles of ethylene oxide per mole of alkyl phenol. The alkyl substituent in such compounds can be derived, for example, from polymerized propylene, diisobutylene and the like. Examples of compounds of this type include nonyl phenol condensed with about 9.5 moles of ethylene oxide per mole of nonyl phenol; dodecylphenol condensed with about 12 moles of ethylene oxide per mole of phenol; dinonyl phenol condensed with about 15 moles of ethylene oxide per mole of phenol and diisooctyl phenol condensed with about 15 moles of ethylene oxide per mole of phenol.

A further class of useful nonionic surfactants include the condensation products of aliphatic alcohols with from about 1 to about 60 moles of an alkylene oxide, especially an ethylene oxide. The alkyl chain of the aliphatic alcohol can either be straight or branched, primary or secondary, and generally contains from about 8 to about 22 carbon atoms. Examples of such ethoxylated alcohols include the condensation product of myristyl alcohol condensed with about 10 moles of ethylene oxide per mole of alcohol and the condensation product of about 9 moles of ethylene oxide with coconut alcohol (a mixture of fatty alcohols with alkyl chains varying in length from about 10 to 14 carbon atoms). Other examples are those C₆-C₁₁ straight-chain alcohols which are ethoxylated with from about 3 to about 6 moles of ethylene oxide. Their derivation is well known in the art. Examples include Alfonic® 810-4.5, which is described in product literature from Sasol as a C₈-C₁₀ straight-chain alcohol having an average molecular weight of 356, an ethylene oxide content of about 4.85 moles (about 60 wt. %), and an HLB of about 12; Alfonic® 810-2, which is described in product literature as a C₉-C₁₀ straight-chain alcohols having an average molecular weight of 242, an ethylene oxide content of about 2.1 moles (about 40 wt. %), and an HLB of about 12; and Alfonic® 610-3.5, which is described in product literature as having an average molecular weight of 276, an ethylene oxide content of about 3.1 moles (about 50 wt. %), and an HLB of 10. Other examples of alcohol ethoxylates are C₁₀ oxo-alcohol ethoxylates available from BASF under the Lutensol® ON tradename. They are available in grades containing from about 3 to about 11 moles of ethylene oxide (available under the names Lutensol®D ON 30; Lutensol® ON 50; Lutensol® ON 60; Lutensol® ON 65; Lutensol® ON 66; Lutensol®D ON 70; Lutensol® ON 80; and Lutensol® ON 110). Other examples of ethoxylated alcohols include the Neodol® 91 series non-ionic surfactants available from Shell Chemical Company which are described as C₉-C₁₁ ethoxylated alcohols. The Neodol® 91 series non-ionic surfactants of interest include Neodol® 91-2.5, Neodol® 91-6, and Neodol® 91-8. Neodol® 91-2.5 has been described as having about 2.5 ethoxy groups per molecule; Neodol 91-6 has been described as having about 6 ethoxy groups per molecule; and Neodol 91-8 has been described as having about 8 ethoxy groups per molecule. Further examples of ethoxylated alcohols include the Rhodasurf® DA series non-ionic surfactants available from Rhodia which are described to be branched isodecyl alcohol ethoxylates. Rhodasurf® DA-530 has been described as having 4 moles of ethoxylation and an HLB of 10.5; Rhodasurf® DA-630 has been described as having 6 moles of ethoxylation with an HLB of 12.5; and Rhodasurf® DA-639 is a 90% solution of DA-630. Further examples of ethoxylated alcohols include those from Tomah Products (Milton, Wis.) under the Tomadol® tradename with the formula RO(CH₂CH₂O)ₙH where R is the primary linear alcohol and n is the total number of moles of ethylene oxide. The ethoxylated alcohol series from Tomah include 91-2.5; 91-6; 91-8—where R is linear C₉/C₁₀/C₁₁ and n is 2.5, 6, or 8; 1-3; 1-5; 1-7; 1-73B; 1-9; where R is linear C₁₁ and n is 3, 5, 7 or 9; 23-1; 23-3; 23-5; 23-6.5—where R is linear C₁₂/C₁₃ and n is 1, 3, 5, or 6.5; 25-3; 25-7; 25-9; 25-12—where R is linear C₁₂/C₁₃/C₁₄/C₁₅ and n is 3, 7, 9, or 12; and 45-7; 45-13—where R is linear C₁₄/C₁₅ and n is 7 or 13.

A further class of useful nonionic surfactants include primary and secondary linear and branched alcohol ethoxylates, such as those based on C₆-C₁₈ alcohols which further include an average of from 2 to 80 moles of ethoxylation per mol of alcohol. These examples include the Genapol® UD (ex. Clariant, Muttenz, Switzerland) described under the tradenames Genapol® UD 030, C₁₁-oxo-alcohol polyglycol ether with 3 EO; Genapol® UD, 050 C₁₁-oxo-alcohol polyglycol ether with 5 EO; Genapol® UD 070, C₁₁-oxo-alcohol polyglycol ether with 7 EO; Genapol® UD 080, C₁₁-oxo-alcohol polyglycol ether with 8 EO; Genapol® UD 088, C₁₁-oxo-alcohol polyglycol ether with 8 EO; and Genapol® UD 110, C₁₁-oxo-alcohol polygycol ether with 11 EO.

Exemplary useful nonionic surfactants include the condensation products of a secondary aliphatic alcohols containing 8 to 18 carbon atoms in a straight or branched chain configuration condensed with 5 to 30 moles of ethylene oxide. Examples of commercially available nonionic detergents of the foregoing type are those presently commercially available under the trade name of Tergitol® such as Tergitol 15-S-12 which is described as being C₁₁-C₁₅ secondary alkanol condensed with 9 ethylene oxide units, or Tergitol 15-S-9 which is described as being C₁₁-C₁₅ secondary alkanol condensed with 12 ethylene oxide units per molecule.

A further class of useful nonionic surfactants include those surfactants having a formula:
RO(CH₂CH₂O)ₙH
wherein;
R is a mixture of linear, even carbon-number hydrocarbon chains ranging from C₁₂H₂₅ to C₁₆H₃₃ and n represents the number of ethoxy repeating units and is a number of from about 1 to about 12.

Surfactants of this formula are presently marketed under the Genapol® tradename (ex. Clariant), which surfactants include the “26-L” series of the general formula RO(CH₂CH₂O)ₙH wherein R is a mixture of linear, even carbon-number hydrocarbon chains ranging from C₁₂H₂₅ to C₁₆H₃₃ and n represents the number of repeating units and is a number of from 1 to about 12, such as 26-L-1,26-L-1.6, 26-L-2,26-L-3,26-L-5, 26-L-45, 26-L-50, 26-L-60, 26-L-60N, 26-L-75, 26-L-80, 26-L-98N, and the 24-L series, derived from synthetic sources and typically contain about 55% C₁₂ and 45% C₁₄ alcohols, such as 24-L-3, 24-L-45, 24-L-50, 24-L-60, 24-L-60N, 24-1-75, 24-L-92, and 24-L-98N, all sold under the Genapol® tradename.

Further useful non-ionic surfactants which may be used in the inventive compositions include those presently marketed under the trade name Pluronics® (ex. BASF). The compounds are formed by condensing ethylene oxide with a hydrophobic base formed by the condensation of propylene oxide with propylene glycol. The molecular weight of the hydrophobic portion of the molecule is of the order of 950 to 4,000 and preferably 200 to 2,500. The addition of polyoxyethylene radicals of the hydrophobic portion tends to increase the solubility of the molecule as a whole so as to make the surfactant water-soluble. The molecular weight of the block polymers varies from 1,000 to 15,000 and the polyethylene oxide content may comprise 20% to 80% by weight. Preferably, these surfactants are in liquid form and particularly satisfactory surfactants are available as those marketed as Pluronics® L62 and Pluronics® L64.

Further nonionic surfactants which may be included in the inventive compositions include alkoxylated alkanolamides, preferably C₈-C₂₄ alkyl di(C₂-C₃ alkanol amides), as represented by the following formula:
R₅—CO—NH—R₆—OH
wherein R₅ is a branched or straight chain C₈-C₂₄ alkyl radical, preferably a C₁₀-C₁₆ alkyl radical and more preferably a C₁₂-C₁₄ alkyl radical, and R₆ is a C₁-C₄ alkyl radical, preferably an ethyl radical.

According to certain particularly preferred embodiments the detersive surfactant constituent necessarily comprises a nonionic surfactant based on a linear primary alcohol ethoxylate particularly wherein the alkyl portion is a C₈ to C₁₆, but particularly a C₉ to C₁₁ alkyl group, and having an average of between about 6 to about 8 moles of ethoxylation.

One further useful class of nonionic surfactants include those in which the major portion of the molecule is made up of block polymeric C₂-C₄ alkylene oxides, with alkylene oxide blocks containing C₃ to C₄ alkylene oxides. Such nonionic surfactants, while preferably built up from an alkylene oxide chain starting group, can have as a starting nucleus almost any active hydrogen containing group including, without limitation, amides, phenols, and secondary alcohols.

One group of nonionic surfactants containing the characteristic alkylene oxide blocks are those which may be generally represented by the formula (A):
HO-(EO)ₓ(PO)_{y}(EO)_{z}—H (A)
where EO represents ethylene oxide,

PO represents propylene oxide,

y equals at least 15,

(EO)_{x+z} equals 20 to 50% of the total weight of said compounds, and,

the total molecular weight is preferably in the range of about 2000 to 15,000.

Another group of nonionic surfactants appropriate for use in the new compositions can be represented by the formula (B):
R-(EO,PO)ₐ(EO,PO)_{b}—H (B)
wherein R is an alkyl, aryl or aralkyl group,
the alkoxy group contains 1 to 20 carbon atoms, the weight percent of EO is within the range of 0 to 45% in one of the blocks a, b, and within the range of 60 to 100% in the other of the blocks a, b, and the total number of moles of combined EO and PO is in the range of 6 to 125 moles, with 1 to 50 moles in the PO rich block and 5 to 100 moles in the EO rich block.

Further nonionic surfactants which in general are encompassed by Formula B include butoxy derivatives of propylene oxide/ethylene oxide block polymers having molecular weights within the range of about 2000-5000.

Still further useful nonionic surfactants containing polymeric butoxy (BO) groups can be represented by formula (C) as follows:
RO—(BO)ₙ(EO)ₓ—H (C)
wherein R is an alkyl group containing 1 to 20 carbon atoms,

n is about 15 and x is about 15.

Also useful as the nonionic block copolymer surfactants which also include polymeric butoxy groups are those which may be represented by the following formula (D):
HO-(EO)ₓ(BO)ₙ(EO)_{y}—H (D)
wherein
n is about 15,
x is about 15 and
y is about 15.

Still further useful nonionic block copolymer surfactants include ethoxylated derivatives of propoxylated ethylene diamine, which may be represented by the following formula: 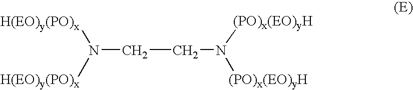
where

(EO) represents ethoxy,

(PO) represents propoxy,

the amount of (PO)ₓ is such as to provide a molecular weight prior to ethoxylation of about 300 to 7500, and the amount of (EO)_{y} is such as to provide about 20% to 90% of the total weight of said compound.

Further useful nonionic surfactants include nonionic amine oxide constituent. Exemplary amine oxides include:

A) Alkyl di(lower alkyl)amine oxides in which the alkyl group has about 10-20, and preferably 12-16 carbon atoms, and can be straight or branched chain, saturated or unsaturated. The lower alkyl groups include between 1 and 7 carbon atoms. Examples include lauryl dimethyl amine oxide, myristyl dimethyl amine oxide, and those in which the alkyl group is a mixture of different amine oxide, dimethyl cocoamine oxide, dimethyl(hydrogenated tallow) amine oxide, and myristyl/palmityl dimethyl amine oxide;

B) Alkyl di(hydroxy lower alkyl)amine oxides in which the alkyl group has about 10-20, and preferably 12-16 carbon atoms, and can be straight or branched chain, saturated or unsaturated. Examples are bis(2-hydroxyethyl)cocoamine oxide, bis(2-hydroxyethyl)tallowamine oxide; and bis(2-hydroxyethyl)stearylamine oxide;

C) Alkylamidopropyl di(lower alkyl)amine oxides in which the alkyl group has about 10-20, and preferably 12-16 carbon atoms, and can be straight or branched chain, saturated or unsaturated. Examples are cocoamidopropyl dimethyl amine oxide and tallowamidopropyl dimethyl amine oxide; and

D) Alkylmorpholine oxides in which the alkyl group has about 10-20, and preferably 12-16 carbon atoms, and can be straight or branched chain, saturated or unsaturated.

Preferably the amine oxide constituent is an alkyl di(lower alkyl)amine oxide as denoted above and which may be represented by the following structure: 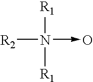
wherein each:

R₁ is a straight chained C₁-C₄ alkyl group, preferably both R₁ are methyl groups; and,

R₂ is a straight chained C₈-C₁₈ alkyl group, preferably is C₁₀-C₁₄ alkyl group, most preferably is a C₁₂ alkyl group.

Each of the alkyl groups may be linear or branched, but most preferably are linear. Most preferably the amine oxide constituent is lauryl dimethyl amine oxide. Technical grade mixtures of two or more amine oxides may be used, wherein amine oxides of varying chains of the R₂ group, are present. Preferably, the amine oxides used in the present invention include R₂ groups which comprise at least 50% wt., preferably at least 60% wt. of C₁₂ alkyl groups and at least 25% wt. of C₁₄ alkyl groups, with not more than 15% wt. of C₁₆, C₁₈ or higher alkyl groups as the R₂ group.

Still further exemplary useful nonionic surfactants which may be used include certain alkanolamides including monoethanolamides and diethanolamides, particularly fatty monoalkanolamides and fatty dialkanolamides.

A cationic surfactant may be incorporated as a germicide or as a detersive surfactant in the solid block composition of the present invention, particularly wherein a bleach constituent is absent from the solid block composition. Cationic surfactants are per se, well known, and exemplary useful cationic surfactants may be one or more of those described for example in McCutcheon's Functional Materials, Vol. 2, 1998; Kirk-Othmer, Encyclopedia of Chemical Technology, 4th Ed., Vol. 23, pp. 481-541 (1997), the contents of which are herein incorporated by reference. These are also described in the respective product specifications and literature available from the suppliers of these cationic surfactants.

Examples of preferred cationic surfactant compositions useful in the practice of the instant invention are those which provide a germicidal effect to the concentrate compositions, and especially preferred are quaternary ammonium compounds and salts thereof, which may be characterized by the general structural formula: 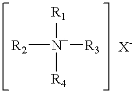
where at least one of R₁, R₂, R₃ and R₄ is a alkyl, aryl or alkylaryl substituent of from 6 to 26 carbon atoms, and the entire cation portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the abovementioned alkyl substituents are hydrocarbons usually containing no more than 12 carbon atoms. The substituents R₁, R₂, R₃ and R₄ may be straight-chained or may be branched, but are preferably straight-chained, and may include one or more amide, ether or ester linkages. The counterion X may be any salt-forming anion which permits water solubility of the quaternary ammonium complex.

Exemplary quaternary ammonium salts within the above description include the alkyl ammonium halides such as cetyl trimethyl ammonium bromide, alkyl aryl ammonium halides such as octadecyl dimethyl benzyl ammonium bromide, N-alkyl pyridinium halides such as N-cetyl pyridinium bromide, and the like. Other suitable types of quaternary ammonium salts include those in which the molecule contains either amide, ether or ester linkages such as octyl phenoxy ethoxy ethyl dimethyl benzyl ammonium chloride, N-(laurylcocoaniinoformylmethyl)-pyridinium chloride, and the like. Other very effective types of quaternary ammonium compounds which are useful as germicides include those in which the hydrophobic radical is characterized by a substituted aromatic nucleus as in the case of lauryloxyphenyltrimethyl ammonium chloride, cetylaminophenyltrimethyl ammonium methosulfate, dodecylphenyltrimethyl ammonium methosulfate, dodecylbenzyltrirethyl ammonium chloride, chlorinated dodecylbenzyltrimethyl ammonium chloride, and the like.

Preferred quaternary ammonium compounds which act as germicides and which are be found useful in the practice of the present invention include those which have the structural formula: 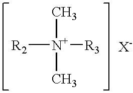
wherein R₂ and R₃ are the same or different C₈-C₁₂alkyl, or R₂ is C₁₂₋₁₆alkyl, C₈₋₁₈alkylethoxy, C₈₋₁₈alkylphenolethoxy and R₃ is benzyl, and X is a halide, for example chloride, bromide or iodide, or is a methosulfate anion. The alkyl groups recited in R₂ and R₃ may be straight-chained or branched, but are preferably substantially linear.

Particularly useful quaternary germicides include compositions which include a single quaternary compound, as well as mixtures of two or more different quaternary compounds. Such useful quaternary compounds are available under the BARDAC®, BARQUAT®, HYAMINE®, LONZABAC®, and ONYXIDE® trademarks, which are more fully described in, for example, McCutcheon's Functional Materials (Vol. 2), North American Edition, 1998, as well as the respective product literature from the suppliers identified below. For example, BARDAC® 205M is described to be a liquid containing alkyl dimethyl benzyl ammonium chloride, octyl decyl dimethyl ammonium chloride; didecyl dimethyl ammonium chloride, and dioctyl dimethyl ammonium chloride (50% active) (also available as 80% active (BARDAC® 208M)); described generally in McCutcheon's as a combination of alkyl dimethyl benzyl ammonium chloride and dialkyl dimethyl ammonium chloride); BARDAC® 2050 is described to be a combination of octyl decyl dimethyl ammonium chloride/didecyl dimethyl ammonium chloride, and dioctyl dimethyl ammonium chloride (50% active) (also available as 80% active (BARDAC® 2080)); BARDAC® 2250 is described to be didecyl dimethyl ammonium chloride (50% active); BARDAC® LF (or BARDAC® LF-80), described as being based on dioctyl dimethyl ammonium chloride (BARQUAT® MB-50, MX-50, OJ-50 (each 50% liquid) and MB-80 or MX-80 (each 80% liquid) are each described as an alkyl dimethyl benzyl ammonium chloride; BARDAC® 4250 and BARQUAT® 4250Z (each 50% active) or BARQUAT® 4280 and BARQUAT 4280Z (each 80% active) are each described as alkyl dimethyl benzyl ammonium chloride/alkyl dimethyl ethyl benzyl ammonium chloride. Also, HYAMINE® 1622, described as diisobutyl phenoxy ethoxy ethyl dimethyl benzyl ammonium chloride (50% solution); HYAMINE®D 3500 (50% actives), described as alkyl dimethyl benzyl ammonium chloride (also available as 80% active (HYAMINE® 3500-80)); and HYMAINE® 2389 described as being based on methyldodecylbenzyl ammonium chloride and/or methyldodecylxylene-bis-triethyl ammonium chloride. (BARDAC®, BARQUAT® and HYAMINE® are presently commercially available from Lonza, Inc., Fairlawn, N.J.). BTC® 50 NF (or BTC® 65 NF) is described to be alkyl dimethyl benzyl ammonium chloride (50% active); BTC® 99 is described as didecyl dimethyl ammonium chloride (50% active); BTC® 776 is described to be myrisalkoniim chloride (50% active); BTC®D 818 is described as being octyl decyl dimethyl ammonium chloride, didecyl dimethyl ammonium chloride, and dioctyl dimethyl ammonium chloride (50% active) (available also as 80% active (BTC® 818-80%)); BTC® 824 and BTC® 835 are each described as being of alkyl dimethyl benzyl ammonium chloride (each 50% active); BTC®D 885 is described as a combination of BTC® 835 and BTC® 818 (50% active) (available also as 80% active (BTC® 888)); BTC® 1010 is described as didecyl dimethyl ammonium chloride (50% active) (also available as 80% active (BTC® 1010-80)); BTC® 2125 (or BTC®D 2125 M) is described as alkyl dimethyl benzyl ammonium chloride and alkyl dimethyl ethylbenzyl ammonium chloride (each 50% active) (also available as 80% active (BTC® 2125 80 or BTC® 2125 NO); BTC® 2565 is described as alkyl dimethyl benzyl ammonium chlorides (50% active) (also available as 80% active (BTC® 2568)); BTC® 8248 (or BTC® 8358) is described as alkyl dimethyl benzyl ammonium chloride (80% active) (also available as 90% active (BTC® 8249)); ONYXIDE® 3300 is described as n-alkyl dimethyl benzyl ammonium saccharinate (95% active). (BTC® and ONYXIDE® are presently commercially available from Stepan Company, Northfield, Ill.) Polymeric quaternary ammonium salts based on these monomeric structures are also considered desirable for the present invention. One example is POLYQUAT®, described as being a 2-butenyldimethyl ammonium chloride polymer.

When present in a solid block composition, it is preferred that the germicidal cationic surfactant(s) are present in amounts so to dispense at least about 200 parts per million (ppm) in the water flushed into the sanitary appliance, e.g., toilet bowl, or into the water retained in the sanitary appliance at the conclusion of the flush cycle.

Further detersive surfactants which may be included are amphoteric and zwitterionic surfactants which provide a detersive effect. Exemplary useful amphoteric surfactants include alkylbetaines, particularly those which may be represented by the following structural formula:
RN⁺(CH₃)₂CH₂COO⁻
wherein R is a straight or branched hydrocarbon chain which may include an aryl moiety, but is preferably a straight hydrocarbon chain containing from about 6 to 30 carbon atoms. Further exemplary useful amphoteric surfactants include amidoalkylbetaines, such as amidopropylbetaines which may be represented by the following structural formula:
RCONHCH₂CH₂CH₂N⁺(CH₃)₂CH₂COO⁻
wherein R is a straight or branched hydrocarbon chain which may include an aryl moiety, but is preferably a straight hydrocarbon chain containing from about 6 to 30 carbon atoms.

As noted above, preferred detersive surfactants are those which exhibit a melting points above about 110° F., preferably above 125° F., in order to permit convenient processing according to known art techniques. Nonetheless small amounts of low melting point surfactants, i.e., those exhibiting melting points below about 110° F. and even liquid surfactants may be used in providing the surfactant constituent of the solid block composition.

The detersive surfactants may be present in any effective amount, however desirably the total amount of detersive surfactants comprise at least 50% wt., preferably at least 55% wt., more preferably at least about 60% wt. of the total weight of the treatment block of which they form a part.

As the performance requirements of treatment blocks may differ according to their use as either an ITB or as an ITC block, the amounts of the constituents present in the block may vary as well depending upon the final intended use of the treatment block.

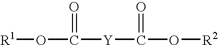

When intended for use as an ITB block, the detersive surfactant constituent may be present in any effective amount and generally comprises up to about 90% wt. of the total weight of the solid block composition, and the resultant treatment block formed therefrom. Preferably the detersive surfactant constituent comprises about 20-90% wt., more preferably 35-80% wt. of the solid block composition, and when used as an ITB block the detersive surfactant constituent most preferably comprises about 50-75% wt. of the solid block composition, and the resultant treatment block formed therefrom. When intended for use as an ITC block, the detersive surfactant constituent may be present in any effective amount and generally comprises up to about 60% wt. of the total weight of the solid block composition, and the resultant treatment block formed therefrom. Preferably the detersive surfactant constituent comprises about 10-55% wt., more preferably 20-50% wt. of the solid block composition, and the resultant treatment block formed therefrom.

As a further essential constituent the solid block composition as well as the treatment blocks formed therefrom necessarily comprise a diester constituent which functions as a useful processing aid in formation of the treatment blocks of the invention. The diester constituent is one or more compounds which may be represented by the following structure:

wherein:

R¹ and R² can independently be C₁-C₆ alkyl which may optionally substituted,

Y is (CH₂)ₓ, wherein x is 0-10, but is preferably 1-8, and while Y may be a linear alkyl or phenyl moiety, desirably Y includes one or more oxygen atoms and/or is a branched moiety.

Exemplary diester constituents include the following diester compounds according to the foregoing structure: dimethyl oxalate, diethyl oxalate, diethyl oxalate, dipropyl oxalate, dibutyl oxalate, diisobutyl oxalate, dimethyl succinate, diethyl succinate, diethylhexyl succinate, dimethyl glutarate, diisostearyl glutarate, dimethyl adipate, diethyl adipate, diisopropyl adipate, dipropyl adipate, dibutyl adipate, diisobutyl adipate, dihexyladipate, di-C₁₂₋₁₅-alkyl adipate, dicapryl adipate, dicetyl adipate, diisodecyl adipate, diisocetyl adipate, diisononyl adipate, diheptylundecyl adipate, ditridecyl adipate, diisostearyl adipate, diethyl sebacate, diisopropyl sebacate, dibutyl sebacate, diethylhexylsebacate, diisocetyl dodecanedioate, dimethyl brassylate, dimethyl phthalate, diethyl phthalate, dibutyl phthalate.

Further exemplary useful diester compounds include those wherein:

Y represents a —CH═CH— moiety such as in dibehenyl fumarate, di-C₁₂₋₁₅ alkyl fumarate, di-C₁₂₋₁₅ alkyl maleate, dicapryl maleate, diethylhexylmaleate, diisostearyl fumarate;

Y represents a —CH(OH)—CH₂— moiety such as in di-C₁₂₋₁₃ alkyl malate and diisostearyl malate;

Y represents a —CH(OH)—CH(OH)— moiety such as in di-C₁₂₋₁₃ alkyl tartrate, di-C₁₄-15 alkyl tartrate and dimyristyl tartrate;

Y represents a —CH₂—CH(SO₃Na)— moiety such as in diamyl sodium sulfosuccinate, dicapryl sodium sulfosuccinate, dicyclohexyl sodium sulfosuccinate, diethylhexyl sodium sulfosuccinate, dihexyl sodium sulfosuccinate, diheptyl sodium sulfosuccinate, diisobutyl sodium sulfosuccinate, and ditridecyl sodium sulfosuccinate;

Y represents a —CH₂—CH(HNCOCH₃)— moiety such as in diethyl acetyl aspartate;

Y represents a —CH₂—CH(NH₂)— moiety such as in diethyl aspartate,

Y represents a —CH₂CH₂CH(NH₂)— moiety such as in diethyl glutamate;

Y represents a —CH₂—CH(HNCO(CH₂)₁₄CH₃)— moiety such as in diethyl palmitoyl aspartate;

Y represents a —C(O)—CH₂—C(O)—CH₂—C(O)— moiety such as in diethyl trioxopimelate;

Y represents a —CH₂—C(OH)(COOH)—CH₂— moiety such as in dilauryl citrate.

Further exemplary useful diester compounds wherein the Y moiety is branched include wherein:

Y represents a —CH₂—C(OH)(COOR)—CH₂— moiety such as in tributyl citrate, triethyl citrate, triisopropyl citrate, triethylhexyl citrate, tri-C₁₂₋₁₃ alkyl citrate, tri-C₁₄₋₁₅ alkyl citrate, tricaprylyl citrate, triisocetyl citrate, trioleyl citrate, tristearyl citrate, triisostearyl citrate, trilauryl citrate, and trioctyldodecyl citrate.

Preferred diester constituents include those wherein Y is —(CH₂)ₓ— wherein x has a value of from 0-6, preferably a value of 0-5, more preferably a value of from 14, while R¹ and R² are C₁-C₆ alkyl groups which may be straight chained alkyl but preferably are branched, e.g, iso- and tert-moieties. Particularly preferred diester compounds are those in which the compounds terminate in ester groups.

Further preferred diester constituents also include those wherein Y represents a moiety selected from: —CH₂—CH(SO₃Na)—, —CH₂—CH(HNCOCH₃)—, —CH₂—CH(NH₂)—, —CH₂CH₂CH(NH₂)—, and —C(O)—CH₂—C(O)—CH₂—C(O)—. Particularly preferred diester compounds are those in which the compounds terminate in ester groups.

The diester constituent may be present in any effective amount and but generally does not exceed about 40% wt. of the total weight of the solid block composition, and the resultant treatment block formed therefrom. Wherein the solid treatment block is intended to be used in an ITB application the preferably the diester constituent comprises about 0.01-20% wt., more preferably from about 2-10% wt. and most preferably from about 2-6% wt. of the solid block composition, and the resultant treatment block formed therefrom. Wherein the solid treatment block is intended to be used in an ITC application the diester constituent comprises to about 40% wt, preferably about 0.01-20% wt., more preferably from about 4-20% wt. and most preferably from about 4-16% wt. of the solid block composition, and the resultant treatment block formed therefrom.

The present inventor has surprisingly found that the inclusion of the diester constituent in the solid block composition provides for improved compositions which may be processed into solid forms, e.g., treatment blocks at lower process temperatures than frequently required of conventional processing aids. The ability to process at lower temperature also provides for the decreased likelihood of the degradation of one or more of the constituents in the solid block compositions during processing, particularly non-halogen releasing constituents which may be deleteriously affected when contacted with the bleach constituent. Further, it is believed that the treatment blocks formed from the inventive compositions exhibit improved physical stability during the usage of the treatment block either as in an ITC or ITB type application.

According to certain and preferred aspects of the invention there is necessarily included a bleach constituent. The bleach constituent is relatively inert in the dry state but, which on contact with water, releases oxygen, hypohalite or a halogen especially chlorine. Representative examples of typical oxygen-release bleaching agents, suitable for incorporation in the solid block composition include the alkali metal perborates, e.g., sodium perborate, and alkali metal monopersulfates, e.g., sodium monopersulfates, potassium monopersulfate, alkali metal monoperphosphates, e.g., disodium monoperphosphate and dipotassium monoperphosphate, as well as other conventional bleaching agents capable of liberating hypohalite, e.g., hypochlorite and/or hypobromite, include heterocyclic N-bromo- and N-chloro-cyanurates such as trichloroisocyanuric and tribromoiscyanuric acid, dibromocyanuric acid, dichlorocyanuric acid, N-monobromo-N-mono-chlorocyanuric acid and N-monobromo-N,N-dichlorocyanuric acid, as well as the salts thereof with water solubilizing cations such as potassium and sodium, e.g., sodium N-monobromo-N-monochlorocyanurate, potassium dichlorocyanurate, sodium dichlorocyanurate, as well as other N-bromo and N-chloro-imides, such as N-brominated and N-chlorinated succinimide, malonimide, phthalimide and naphthalimide. Also useful in the solid block composition as hypohalite-releasing bleaches are halohydantoins which may be used include those which may be represented by the general structure: 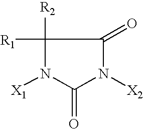
wherein:

X₁ and X₂ are independently hydrogen, chlorine or bromine; and,

R₁ and R₂ are independently alkyl groups having from 1 to 6 carbon atoms. Examples of halohydantoins include, for example, N,N′-dichloro-dimethyl-hydantoin, N-bromo-N-chloro-dimethyl-hydantoin, N,N′-dibromo-dimethyl-hydantoin, 1,4-dichloro, 5,5-dialkyl substituted hydantoin, wherein each alkyl group independently has 1 to 6 carbon atoms, N-monohalogenated hydantoins such as chlorodimethylhydantoin (MCDMH) and N-bromo-dimethylhydantoin (MBDMH); dihalogaenated hydantoins such as dichlorodimethylhydantoin (DCDMH), dibromodimethylhydantoin (DBDMW), and 1-bromo-3-chloro-5,5,-dimethylhydantoin (BCDMH); and halogenated methylethylhydantoins such as chloromethylethylhydantion (MCMEH), dichloromethylethylhydantoin (DCMEH), bromomethylethylhydantoin (MBMEH), dibromomethylethylhydantoin (DBMEH), and bromochloromethylethylhydantoin (BCMEH), and mixtures thereof. Other suitable organic hypohalite liberating bleaching agents include halogenated melamines such as tribromomelamine and trichloromelamine. Suitable inorganic hypohalite-releasing bleaching agents include lithium and calcium hypochlorites and hypobromites. The various chlorine, bromine or hypohalite liberating agents may, if desired, be provided in the form of stable, solid complexes or hydrates, such as sodium p-toluene sulfobromamine trihydrate; sodium benzene sulfochloramine dihydrate; calcium hypobromite tetrahydrate; and calcium hypochlorite tetrahydrate. Brominated and chlorinated trisodium phosphates formed by the reaction of the corresponding sodium hypohalite solution with trisodium orthophosphate (and water, as necessary) likewise comprise useful inorganic bleaching agents for incorporation into the inventive solid block composition and the treatment blocks formed therefrom.

Preferably, the bleach constituent necessarily present according to the second aspect of the solid block composition of the invention is a hypohalite liberating compound and more preferably is a hypohalite liberating compound in the form of a solid complex or hydrate thereof. Particularly preferred for use as the bleach constituent are chloroisocynanuric acids and alkali metal salts thereof, preferably potassium, and especially sodium salts thereof. Examples of such compounds include trichloroisocyananuric acid, dichloroisocyanuric acid, sodium dichloroisocyanurate, potassium dichloroisocyanurate, and trichloro-potassium dichloroisocynanurate complex. The most preferred chlorine bleach material is sodium dichloroisocyanurate; the dihydrate of this material is particularly preferred.

The bleach constituent may be present in any effective amount and may comprise up to about 90% wt. of the solid block composition and the resultant treatment block formed therefrom. Preferably however the bleach constituent comprises at least about 0.1-60% wt. of the total weight of the solid block composition, and the resultant treatment block formed therefrom, irregardless of use as an ITC or ITB type treatment block. More preferably the bleach constituent comprises about 0.5-50% wt., more preferably at least 1-40% wt. of the solid block composition.

While the solid block composition of the present invention can be made up entirely of the surfactant constituent, the diester compound, and optionally the bleach constituent, in most instances it is nonetheless highly desirable to include additional constituents in the solid block composition. Other constituents may be incorporated into the blocks of the invention as long as they do not adversely affect the properties of the treatment block formed from the solid block composition. It will be noted that for several of the optional constituents as described below, interaction of the components with hypochlorite bleaches, or stability of the components with respect to hypochlorite bleaches are to be considered with respect to the selection of suitable constituents which may be included in the solid block composition.

The inventive solid block compositions may include one or more colorants used to impart a color to the solid block composition, or to the water with which the solid block composition contacts or both. Exemplary useful colorants include any materials which may provide a desired coloring effect. Exemplarly useful coloring agents include dyes, e.g., Alizarine Light Blue B (C.I. 63010), Carta Blue VP (C.I. 24401), Acid Green 2G (C.I. 42085), Astragon Green D (C.I. 42040) Supranol Cyanine 7B (C.I. 42675), Maxilon Blue 3RL (C.I. Basic Blue 80), acid yellow 23, acid violet 17, a direct violet dye (Direct violet 51), Drimarine Blue Z-RL (C.I. Reactive Blue 18), Alizarine Light Blue H-RL (C.I. Acid Blue 182), FD&C Blue No. 1, FD&C Green No. 3 and Acid Blue No. 9. When a bleach constituent is included in the solid block composition, the colorant, e.g., dye, should be selected so to ensure the compatibility of the colorant with the bleach constituent, or so that its color persists despite the presence in the toilet bowl of a concentration of hypochlorite which is effective to maintain sanitary conditions. Frequently however, a solid block composition which includes a bleach constituent do not comprise any colorants. Desirably the colorants, when present, do not exceed 15% wt. of the solid block composition, although generally lesser amounts are usually effective.

The solid block composition of the invention may include one or more perfumes which impart desirable scent characteristics to the solid blocks formed from the solid block composition taught herein. Exemplary perfumes may be any material giving an acceptable odor and thus materials giving a “disinfectant” odor such as essential oils, pine extracts, terpinolenes, ortho phenyl phenol or paradichlorobenzene may be employed. The essential oils and pine extracts also contribute as plasticizers and are functional to a degree in extending block life. The perfume may be in solid form and is suitably present in an amount up to 10% by weight of the solid block composition.

Exemplary, albeit optional constituents are stain inhibiting materials. The solid block composition of the invention may, for example, include an effective amount of a manganese stain inhibiting agent which is advantageously included wherein the sanitary appliance is supplied by a water source having an appreciable or high amount of manganese. Such water containing a high manganese content are known to frequently deposit unsightly stains on surfaces of sanitary appliances, especially when the solid block composition also contains a bleach source which provides a hypochlorite. To counteract such an effect the solid block composition of the present invention may comprise a manganese stain inhibiting agent, such as a partially hydrolyzed polyacrylamide having a molecular weight of about 2000 to about 10,000, a polyacrylate with a molecular weight of about 2000 to about 10,000, and/or copolymers of ethylene and maleic acid anhydride with a molecular weight of from about 20,000 to about 100,000. When present the satin inhibiting materials may comprise to about 10% wt.

The solid block composition of the invention may include a germicide. Exemplary suitable germicides include, for example, formaldehyde release agents, chlorinated phenols, as well as iodophors. It is to be understood that certain cationic surfactants including quaternary ammonium compound based surfactants may also provide a germicidal benefit and may be used in place of the optional further germicide constituent recited here. Further exemplary useful germicides which may be included include methylchloroisothiazolinone/methylisothiazolinone sodium sulfite, sodium bisulfite, imidazolidinyl urea, diazolidinyl urea, benzyl alcohol, 2-bromo-2-nitropropane-1,3-diol, formalin (formaldehyde), iodopropenyl butylcarbamate, chloroacetamide, methanamine, methyldibromonitrile glutaronitrile, glutaraldehyde, 5-bromo-5-nitro-1,3-dioxane, phenethyl alcohol, o-phenylphenol/sodium o-phenylphenol, sodium hydroxymethylglycinate, polymethoxy bicyclic oxazolidine, dimethoxane, thimersal dichlorobenzyl alcohol, captan, chlorphenenesin, dichlorophene, chlorbutanol, glyceryl laurate, halogenated diphenyl ethers, phenolic compounds, mono- and poly-alkyl and aromatic halophenols, resorcinol and its derivatives, bisphenolic compounds, benzoic esters (parabens), halogenated carbanilides, 3-trifluoromethyl-4,4′-dichlorocarbanilide, and 3,31,4-trichlorocarbanilide. More preferably, the non-cationic antimicrobial agent is a mono- and poly-alkyl and aromatic halophenol selected from the group p-chlorophenol, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benzyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol 2-diethylmethyl-3,5-dimethyl p-chlorophenol, 6-sec-octyl-3-methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol, 4-chloro-2-methyl phenol, 4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol, para-chloro-meta-xylenol, dichloro meta xylenol, chlorothymol, and 5-chloro-2-hydroxydiphenylmethane.

When present the germicide is included in the solid block composition in germicidally effective amounts, generally in amounts of up to about 25% wt. of the solid block composition, although generally lesser amounts are usually effective.

A further optional constituent are one or more preservatives. Such preservatives are primarily included to reduce the growth of undesired microorganisms within the treatment blocks formed from the solid block composition during storage prior to use or while used, although it is expected that the such a preservative may impart a beneficial antimicrobial effect to the water in the sanitary appliance to which the treatment block is provided. Exemplary useful preservatives include compositions which include parabens, including methyl parabens and ethyl parabens, glutaraldehyde, formaldehyde, 2-bromo-2-nitropropoane-1,3-diol, 5-chloro-2-methyl-4-isothiazolin-3-one, 2-methyl-4-isothiazoline-3-one, and mixtures thereof. One exemplary composition is a combination 5-chloro-2-methyl-4-isothiazolin-3-one and 2-methyl-4-isothiazolin-3-one where the amount of either component may be present in the mixture anywhere from 0.001 to 99.99 weight percent, based on the total amount of the preservative. For reasons of availability, the most preferred preservative are those commercially available preservative comprising a mixture of 5-chloro-2-methyl-4-isothiazolin-3-one and 2-methyl-4-isothiazolin-3-one marketed under the trademark KATHON® CG/ICP as a preservative composition presently commercially available from Rohm and Haas (Philadelphia, Pa.). Further useful preservative compositions include KATHON® CG/ICP II, a further preservative composition presently commercially available from Rohm and Haas (Philadelphia, Pa.), PROXEL® which is presently commercially available from Zeneca Biocides (Wilmington, Del.), SUTTOCIDE® A which is presently commercially available from Sutton Laboratories (Chatam, N.J.) as well as TEXTANMR® 38AD which is presently commercially available from Calgon Corp. (Pittsburgh, Pa.). When present, the optional preservative constituent should not exceed about 5% wt. of the solid block composition, although generally lesser amounts are usually effective.

The inventive solid block composition may include a binder constituent. The binder may function in part controlling the rate of dissolution of the tablet. The binder constituent may be a clay, but preferably is a water-soluble or water-dispersible gel-forming organic polymer. The term “gel-forming” as applied to this polymer is intended to indicate that on dissolution or dispersion in water it first forms a gel which, upon dilution with further water, is dissolved or dispersed to form a free-flowing liquid. The organic polymer serves essentially as binder for the tablets produced in accordance with the invention although, as will be appreciated, certain of the polymers envisaged for use in accordance with the invention also have surface active properties and thereby serve not only as binders but also enhance the cleansing ability of the tablets of the invention. Further certain organic polymers, such as substituted celluloses, also serve as soil antiredeposition agents. A wide variety of water-soluble organic polymers are suitable for use in the solid block composition of the present invention. Such polymers may be wholly synthetic or may be semi-synthetic organic polymers derived from natural materials. Thus, for example, on class of organic polymers for use in accordance with the invention are chemically modified celluloses such as ethyl cellulose, methyl cellulose, sodium carboxymethyl cellulose, hydroxypropyl cellulose, hydroxypropyl methyl cellulose, ethyl hydroxyethyl cellulose, carboxymethyl hydroxyethyl cellulose, and hydroxyethyl cellulose. Another class of organic polymers which may be used include naturally derived or manufactured (fermented) polymeric materials such as alginates and carageenan. Also, water-soluble starches and gelatin may be used as the optional binder constituent. The cellulose based binders are a preferred class of binders for use in the solid block composition and may possess the property of inverse solubility that is their solubility decreases with increasing temperature, thereby rendering the tablets of the invention suitable for use in locations having a relatively high ambient temperature.

The optional binder constituent may also be one or more synthetic polymers e.g, polyvinyl alcohols; water-soluble partially hydrolyzed polyvinyl acetates; polyacrylonitriles; polyvinyl pyrrolidones; water-soluble polymers of ethylenically unsaturated carboxylic acids, such as acrylic acid and methacrylic acid, and salts thereof; base-hydrolysed starch-polyacrylonitrile copolymers; polyacrylamides; ethylene oxide polymers and copolymers; as well as carboxypolymethylenes.

In the case of the organic polymeric binders it may be noted that, in general, the higher the molecular weight of the polymer the greater the in-use life of the treatment block of the invention. When present, the total binder content may comprise up to 75% wt. of the solid block composition, but preferably is from 0.5 to 70% by weight, preferably from 1 to 65% by weight, more preferably from 5 to 60% by weight.

The solid block composition may optionally include one or more dissolution control agents. Such dissolution control agent are materials which provide a degree of hydrophobicity to the treatment block formed from the solid block composition whose presence in the treatment block contributes to the slow uniform dissolution of the treatment block when contacted with water, and simultaneously the controlled release of the active constituents of the solid block composition. Preferred for use as the dissolution control agents are mono- or di-alkanol amides derived from C₈-C₁₆ fatty acids, especially C₁₂-C₁₄ fatty acids having a C₂-C₆ monoamine or diamine moiety. When included the dissolution control agent may be included in any effective amount, but desirably the dissolution control agent is present in an amount not to exceed about 600% wt. of the solid block composition, although generally lesser amounts are usually effective. Generally wherein the treatment block is to be used in an ITB application the dissolution control agent is present to about 12% wt., more preferably is present from 0.1-10% wt. and most preferably is present from about 3-8% wt. of the solid block compositions, as well as in the treatment blocks formed therefrom. Generally wherein the treatment block is to be used in an ITC application the dissolution control agent is present to about 50% wt., more preferably is present from 1-50% wt. and most preferably is present from about 10-40% wt. of the solid block compositions, as well as in the treatment blocks formed therefrom.

The solid block composition may optionally include one or more water-softening agents or one or more chelating agents, for example inorganic water-softening agents such as sodium hexametaphosphate or other alkali metal polyphosphates or organic water-softening agents such as ethylenediaminetetraacetic acid and nitrilotriacetic acid and alkali metal salts thereof. When present, such water-softening agents or chelating agents should not exceed about 20% wt. of the solid block composition, although generally lesser amounts are usually effective.

The solid block composition may optionally include one or more solid water-soluble acids or acid-release agents such as sulphamic acid, citric acid or sodium hydrogen sulphate. When present, such solid water-soluble acids or acid-release agents should not exceed about 20% wt. of the solid block composition, although generally lesser amounts are usually effective.

Diluent materials may be included to provide additional bulk of the product solid block composition and may enhance leaching out of the surfactant constituent when the solid block composition is placed in water. Exemplary diluent materials include any soluble inorganic alkali, alkaline earth metal salt or hydrate thereof, for example, chlorides such as sodium chloride, magnesium chloride and the like, carbonates and bicarbonates such as sodium carbonate, sodium bicarbonate and the like, sulfates such as magnesium sulfate, copper sulfate, sodium sulfate, zinc sulfate and the like, borax, borates such as sodium borate and the like, as well as others known to the art but not particularly recited herein. Exemplary organic diluents include, inter alia, urea, as well as water soluble high molecular weight polyethylene glycol and polypropylene glycol. When present, such diluent materials should not exceed about 40% wt. of the solid block composition, although generally lesser amounts are usually effective.

The solid block composition and treatment blocks formed therefrom may include one or more fillers. Such fillers are typically particulate solid water-insoluble materials which may be based on inorganic materials including but not limited to talc, fumed silica, quartz, pumice, pumicite, titanium dioxide, silica sand, calcium carbonate, zirconium silicate, diatomaceous earth, whiting, feldspar, perlite and expanded perlite. Organic filler materials may also be used, including but not limited to particulate organic polymeric materials such as finely comminuted water insoluble synthetic polymers. When present, such fillers should not exceed about 30% wt., preferably should not exceed about 20% wt. of the solid block composition, although generally lesser amounts are usually effective.

The solid block composition and treatment blocks formed therefrom may include one or more further processing aids. For example, the solid block composition may also include other binding and/or plasticizing ingredients serving to assist in the manufacture thereof, for example, polypropylene glycol having a molecular weight from about 300 to about 10,000 in an amount up to about 20% by weight, preferably about 4% to about 15% by weight of the mixture may be used. The polypropylene glycol reduces the melt viscosity, acts as a demolding agent and also acts to plasticize the block when the composition is prepared by a casting process. Other suitable plasticizers such as pine oil fractions, d-limonene, dipentene and the ethylene oxide-propylene oxide block copolymers may be utilized. Other useful processing aids include tabletting lubricants such as metallic stearates, stearic acid, paraffin oils or waxes or sodium borate which facilitate in the formation of the treatment blocks in a tabletting press or die. When present such further processing aids are typically included in amounts of up to about 10% by weight of the solid block composition, although generally lesser amounts are usually effective.

Ideally the treatment blocks formed from the solid block composition exhibit a density greater than that of water which ensures that they will sink when suspended in a body of water, e.g., the water present within a cistern. Preferably the treatment blocks formed from the solid block composition exhibit a density in excess of about 1 g/cc of water, preferably a density in excess of about 1.5 g/cc of water and most preferably a density of at least about 2 g/cc of water.

The treatment blocks according to the present invention may also be provided with a coating of a water-soluble film, such as polyvinyl acetate following the formation of the treatment blocks from the recited solid block composition. Such may be desired for improved handling, however such is often unnecessary as preferred embodiments of the treatment blocks exhibit a lower likelihood of sticking to one another following manufacture than many prior art treatment block compositions.

The treatment blocks formed from the solid block composition may be used with or without an ancillary device or structure. In one manner of use one or more treatment blocks are supplied to the cistern of a toilet where they sink and typically rest upon the bottom until they are consumed. In another manner of use one or more treatment blocks are supplied to the interior of a sanitary appliance, e.g., a toilet bowl or interior of a urinal wherein the treatment block(s) are within the path of flush water flushed through the sanitary appliance during its normal manner of use.

The manufacture of the solid treatment blocks from the solid block composition according to the present invention is well within the capability of persons of ordinary skill in the art. Exemplary useful processes contemplate by mixing the included constituents into a homogeneous mass and noodling, plodding, extruding, cutting and stamping the mass to form uniform bars or cakes. The constituents ultimately present in the solid blocks are preferably formed by tabletting, casting or extrusion using known techniques. Most preferably solid blocks are conveniently and preferably made by extrusion. Usually all of the solid ingredients are mixed in any suitable blending equipment followed by the addition of liquid ingredients under blending conditions. The resulting homogeneous blend is then extruded.

The blocks of the invention are conveniently formed by a compression process, especially an extrusion process comprising the steps of forming a mixture of the components of the composition, extruding this mixture into rod or bar form and then cutting the extruded rod or bar into appropriately sized pieces or blocks. Typically, the treatment blocks of the present invention weigh from 25 to 150 grams, preferably from about 25 to about 75 grams. The blocks are typically cylindrical in shape, having a length of from about ½ to about 2 inches and having a diameter of about 1 to about 3 inches.

The service life of the treatment blocks should be from about 30 to about 90 days when installed in a toilet tank, based on normal use. The length of life of the product blocks will depend on a variety of factors including product formulation, water temperature, tank size, and the number of flushes over the period of use.

In order to further illustrate the present invention, various examples including preferred embodiments of the invention are described amongst the examples. In these examples, as well as throughout the balance of this specification and claims, all parts and percentages are by weight unless otherwise indicated.

### EXAMPLES

Treatment blocks according to the invention were produced from solid block compositions described on Table 1, following:

TABLE 1

| | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| dodecylbenzene sulfonate, sodium salt (80%) | 55.85 | 58.85 | 62.51 | 62.51 | 58.61 | 67.27 | 69.25 | 70.83 | 69.0 |
| lauramide monoethanolamide (98%) | 6.01 | 6.01 | 6.38 | 6.38 | 5.98 | 4.74 | 4.88 | 4.99 | 1.0 |
| sodium sulfate | 12 | 12 | 12.75 | 12.75 | 11.95 | 17.37 | 17.88 | 18.29 | 24.5 |
| silica | 2.41 | 2.41 | 2.56 | 2.56 | 2.40 | 1.91 | 1.96 | 2.01 | 2.0 |
| dichlorocyanurate dihydrate, sodium salt (56%) | 14.63 | 14.63 | 9.32 | 9.32 | 14.6 | 4.98 | 2.41 | 0.55 | 2.5 |
| diisopropyl adipate | 6.1 | 6.1 | 6.48 | 6.48 | 6.46 | 3.73 | 3.61 | 3.33 | 2.0 |
| | Ex. 10 | Ex. 11 | Ex. 12 | Ex. 13 | Ex.14 | Ex. 15 | Ex. 16 | Ex.17 | |
| dodecylbenzene sulfonate, sodium salt (80%) | 36 | 36 | 28 | 24 | 36 | 68 | 48 | — | |
| C₁₆-C₁₈ olefin sulfonate, sodium salt (80%) | 32 | 32 | 40 | 44 | 32 | — | — | — | |
| lauryl sulfate, sodium salt (93%) | — | — | — | — | — | — | 20 | 20 | |
| secondary alkane sulfonate sodium salt (93%) | — | — | — | — | — | — | — | 48 | |
| lauramide monoethanolamide (98%) | 5 | 5 | 5 | 5 | 5 | — | — | — | |
| sodium sulfate | 16.5 | 18.5 | 18.5 | 18.5 | 18.5 | 18.5 | 18.5 | 18.5 | |
| silica | 2 | 2 | 2 | 2 | 2 | — | — | — | |
| perlite | — | — | — | — | — | 5 | 5 | 5 | |
| dichlorocyanurate dihydrate, sodium salt (56%) | 2.5 | 2.5 | 2.5 | 2.5 | 2.5 | 2.5 | 2.5 | 2.5 | |
| diisopropyl adipate | 6 | 4 | 4 | 4 | 4 | 6 | 6 | 6 | |

| | Ex. 18 | Ex. 19 | Ex. 20 |
|---|---|---|---|
| dodecylbenzene sulfonate, sodium salt (80%) | 48 | — | 37 |
| C₁₆-C₁₈ olefin sulfonate, sodium salt (80%) | — | — | 24 |
| lauryl sulfate, sodium salt (93%) | — | — | — |
| secondary alkane sulfonate, sodium salt (93%) | — | 48 | — |
| diooctyl sulfosuccinate, sodium salt (85%) | 20 | 20 | — |
| lauramide monoethanolamide (98%) | — | — | 12 |
| sodium sulfate | 18.5 | 18.5 | 19.5 |
| silica | — | — | 2 |
| perlite | 5 | 5 | — |
| dichlorocyanurate dihydrate, sodium salt (56%) | 2.5 | 2.5 | 2.5 |
| diisopropyl adipate | 6 | 6 | 3 |

The identity of the constituents used to form the treatment blocks are identified more specifically on the following Table 2.

TABLE 2

| | |
|---|---|
| dodecylbenzene sulfonate, sodium salt (80%) | anionic surfactant, dodecylbenzene |
| | sulfonate, 80% wt. actives |
| C₁₆-C₁₈ olefin sulfonate, sodium salt (80%) | anionic surfactant, C₁₆-C₁₈ olefin |
| | sulfonate, sodium salt, 80% wt. actives |
| lauryl sulfate, sodium salt (93%) | lauryl sulfate, sodium salt, 93% wt. |
| | actives |
| secondary alkane sulfonate, sodium salt (93%) | secondary C₁₄-C₁₇ alkyl sulfonate, |
| | sodium salt, 93% wt. actives |
| diooctyl sulfosuccinate, sodium salt (85%) | diooctyl sulfosuccinate, sodium salt, |
| | 85% wt. actives |
| lauramide monoethanolamide (98%) | solubility control agent, lauramide |
| | monoethanolamide, 98% wt. actives |
| sodium sulfate | diluent, anhydrous sodium sulfate |
| | 100% wt. actives |
| silica | filler anhydrous silica, 100% wt. actives. |
| perlite | perlite, 100% wt. actives |
| dichlorocyanurate dihydrate, sodium salt (56%) | bleach constituent, dichlorocyanurate |
| | dihydrate, sodium salt, 56% wt. bleach |
| | actives |
| diisopropyl adipate | diester constituent, diisopropyl adipate, |
| | 100% wt. actives |

Treatment blocks were formed in accordance with the following general process:

All of the anhydrous constituents, excluding the bleach constituent are dry blended to form a premixture, which is subsequently metered concurrently with appropriate metered amounts of the bleach constituent into the throat of a twin-screw extruder. The twin-screw extruder is operated at low temperatures and pressures, and during mixing metered amounts of the diester constituent is injected into the extruder barrel at a port located about one-third of the distance of the length of the extruder barrel downstream of the throat. The twin-screw extruder is used to form a homogeneous blend of the solid block constituents. Subsequently the exiting homogenous blend exiting the twin-screw extruder is supplied to the throat of s single screw extruder which is used to compress the homogenous blend into a solid mass. The single screw extruder operates at a rotational rate of between 5 rpm and 45 rpm, at a temperature of about 30-50° C., and the extruded solid mass exits a circular die having a diameter in the range of 30-45 millimeters heated to about 40-75° C. Upon exiting the circular die, the solid mass is cut into short cylindrical blocks having an approximate mass of between about 30-40 grams.

The treatment blocks exhibit good dimensional stability both after manufacture and prior to use in the cleaning treatment of a sanitary appliance, e.g., a toilet or urinal, as well as during the cleaning treatment of a sanitary appliance.

While the invention is susceptible of various modifications and alternative forms, it is to be understood that specific embodiments thereof have been shown by way of example in the drawings which are not intended to limit the invention to the particular forms disclosed; on the contrary the intention is to cover all modifications, equivalents and alternatives falling within the scope and spirit of the invention as expressed in the appended claims.

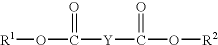

1. A treatment block formed from a solid block composition which includes: a surfactant constituent, a diester constituent, and one or more further optional constituents.

2. A treatment block formed from a solid block composition which includes: a surfactant constituent, a diester constituent, a bleach constituent, and optionally one or more further constituents.

3. A treatment block according to claim 1 or claim 2 wherein the diester constituent a compound which may be represented by the following structure:
wherein:
R¹ and R² can independently be C₁-C₆ alkyl which may optionally substituted, Y is (CH₂)ₓ, wherein x is 0-10, but is preferably 4-8, and while Y may be a linear alkyl or phenyl moiety, desirably Y includes one or more oxygen atoms or is a branched moiety.

4. A treatment block according to claim 3 wherein:
Y is —(CH₂)ₓ— wherein x has a value of from 1-4.

5. A treatment block according to claim 3 wherein:
R¹ and R² are C₁-C₆ alkyl groups.

6. A treatment block according to claim 1 or claim 2 wherein the diester constituent a compound which may be represented by the following structure: 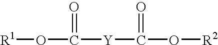
wherein:
R¹ and R² can independently be C₁-C₆ alkyl which may optionally substituted,
Y represents a —CH₂—CH(SO₃Na)— moiety.

7. A treatment block according to claim 1 or claim 2 wherein the diester constituent a compound which may be represented by the following structure: 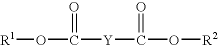
wherein:
R¹ and R² can independently be C₁-C₆ alkyl which may optionally substituted,
Y represents a —CH₂—CH(HNCOCH₃)— moiety.

8. A treatment block according to claim 1 or claim 2 wherein the diester constituent a compound which may be represented by the following structure: 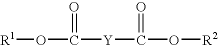
wherein:
R¹ and R² can independently be C₁-C₆ alkyl which may optionally substituted,
Y represents a —CH₂—CH(NH₂)— moiety.

9. A treatment block according to claim 1 or claim 2 wherein the diester constituent a compound which may be represented by the following structure: 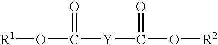
wherein:
R¹ and R² can independently be C₁-C₆ alkyl which may optionally substituted,
Y represents a —CH₂CH₂CH(NH₂)— moiety.

10. A treatment block according to claim 1 or claim 2 wherein the diester constituent a compound which may be represented by the following structure: 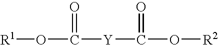
wherein:
R¹ and R² can independently be C₁-C₆ alkyl which may optionally substituted,
Y represents a —C(O)—CH₂—C(O)—CH₂—C(O)— moiety.
